(19) United States
(12) Patent Application Publication
(10) Pub. No.: US 2022/0207393 A1
(43) Pub. Date: Jun. 30, 2022

(54) METHOD OF PREDICTING SEMICONDUCTOR MATERIAL PROPERTIES AND METHOD OF TESTING SEMICONDUCTOR DEVICE USING THE SAME

(71) Applicant: Samsung Electronics Co., Ltd., Suwon-si (KR)

(72) Inventors: Naoto Umezawa, Seongnam-si (KR); Changwook Jeong, Hwaseong-si (KR); Jisu Ryu, Hwaseong-si (KR); Kyu Hyun Lee, Suwon-si (KR); Jinyoung Lim, Seoul (KR); Wonik Jang, Suwon-si (KR); In Huh, Seoul (KR)

(21) Appl. No.: 17/468,819

(22) Filed: Sep. 8, 2021

(30) Foreign Application Priority Data
Dec. 30, 2020 (KR) 10-2020-0187266

(51) Int. Cl. G06N 5/04; G06N 5/02

(52) U.S. Cl. CPC G06N 5/04; G06N 5/02

(57) ABSTRACT

Disclosed are methods of predicting semiconductor material properties and methods of testing semiconductor devices using the same. The prediction method comprises preparing a machine learning model that is trained with a training system and using the machine learning model to predict material properties of a target system. The machine learning model is represented as a function of material properties with respect to a descriptor. The descriptor is calculated from unrelaxed charge density (UCD) that is represented by summation of atomic charge density (ACD) of single atoms.

### CROSS-REFERENCE TO RELATED APPLICATION

This U.S. nonprovisional application claims priority under 35 U.S.C § 119 to Korean Patent Application No. 10-2020-0187266 filed on Dec. 30, 2020 in the Korean Intellectual Property Office, the disclosure of which is hereby incorporated by reference in its entirety.

### FIELD

The present inventive concepts relate to semiconductor material properties, and more particularly, to methods of predicting semiconductor material properties and methods of testing a semiconductor device using the same.

### BACKGROUND

With the high integration and compactness of semiconductor devices, an increase in influence of interaction between single atoms may benefit from an atomic scale modeling approach. Such atomic scale modeling can be accomplished by simulation based on wave nature of electron, or Schrodinger equation.

An ab initio simulation may refer to an atomic scale simulation that may rely only on principles of quantum mechanics and may be free from experimental variables and empirical nature. The ab initio simulation may be a powerful tool to study material properties and may be widely used for material modeling. However, when sufficiently accurate electronic structures are needed, the typical size with which the ab initio simulation can deal may be relatively small (e.g., about 100 atoms) and thus studies have been conducted to expand the capability of the ab initio simulation.

Meanwhile, machine learning may be used in conjunction with the ab initio simulation, and various research has demonstrated the potential of machine learning accelerated by ab initio simulation.

### SUMMARY

Some embodiments of the present inventive concepts provide methods of predicting semiconductor material properties with improved speed and accuracy.

Some example embodiments of the present inventive concepts provide methods of predicting semiconductor material properties for large scale systems.

Some embodiments of the present inventive concepts provide methods of testing semiconductor devices using methods of predicting semiconductor material properties.

The present inventive concepts are not limited to those mentioned above, and other embodiments will be clearly understood to those skilled in the art from the following description.

According to some example embodiments of the present inventive concepts, a method of predicting semiconductor material properties may comprise executing, by at least one processor, commands stored in a non-transitory memory to perform operations comprising: accessing a machine learning model that is trained with a training system, where the training system comprises calculated semiconductor material properties; and using the machine learning model to predict material properties of a target system comprising one or more semiconductor materials. The machine learning model is represented by a function of material properties with respect to a descriptor. The descriptor is calculated from unrelaxed charge density (UCD) that is represented by summation of atomic charge density (ACD) of single atoms

According to some example embodiments of the present inventive concepts, a method of predicting semiconductor material properties may comprise executing, by at least one processor, commands stored in a non-transitory memory to perform operations comprising: accessing a machine learning model that is trained with a training system, where the training system comprises calculated semiconductor material properties generated by an ab initio simulation; fitting the machine learning model to an output of the ab initio simulation; and using the machine learning model to predict material properties of a target system comprising one or more semiconductor materials. The machine learning model is represented by a function of material properties with respect to a descriptor. The descriptor is calculated from unrelaxed charge density (UCD).

According to some example embodiments of the present inventive concepts, a method of testing a semiconductor device may comprise executing, by at least one processor, commands stored in a non-transitory memory to perform operations comprising: accessing a machine learning model that is trained with a training system, where the training system comprises calculated semiconductor material properties generated by density functional theory (DFT); fitting the machine learning model to a result calculated from the density functional theory (DFT); using the machine learning model to predict material properties of a target system that is a portion of the semiconductor device; and testing the semiconductor device based on the material properties that were predicted for the target system by the machine learning model. The machine learning model is represented by a function of material properties with respect to a descriptor. The descriptor is calculated from unrelaxed charge density (UCD) that is represented by summation of atomic charge density (ACD) of single atoms

### BRIEF DESCRIPTION OF THE DRAWINGS

FIG. 1 illustrates a flow chart showing a method of predicting semiconductor material properties according to some embodiments of the present inventive concepts.

FIGS. 2 and 3 illustrate conceptual diagrams showing a training system for a method of predicting semiconductor material properties according to some embodiments of the present inventive concepts.

FIGS. 4 and 5 illustrate graphs showing a calculation procedure in a method of predicting semiconductor material properties according to some embodiments of the present inventive concepts.

FIGS. 6 and 7 illustrate conceptual diagrams showing operations for preparing a machine learning model for a method of predicting semiconductor material properties according to some embodiments of the present inventive concepts.

FIG. 8 illustrates a conceptual diagram showing a target system for a method of predicting semiconductor material properties according to some embodiments of the present inventive concepts.

FIG. 9 illustrates a conceptual diagram showing operations for prediction of material properties of a target system with the help of machine learning model for a method of predicting semiconductor material properties according to some embodiments of the present inventive concepts.

FIGS. 10A, 10B, 11A, 11B, 12A, and 12B illustrate graphs showing errors generated in using a descriptor for a method of predicting semiconductor material properties according to some embodiments of the present inventive concepts.

FIG. 13 illustrates a block diagram showing a computing system related to a method of predicting semiconductor material properties according to some embodiments of the present inventive concepts.

### DETAILED DESCRIPTION OF EMBODIMENTS

The following will now describe methods of predicting semiconductor material properties and methods of testing semiconductor devices using the same with reference to the accompanying drawings.

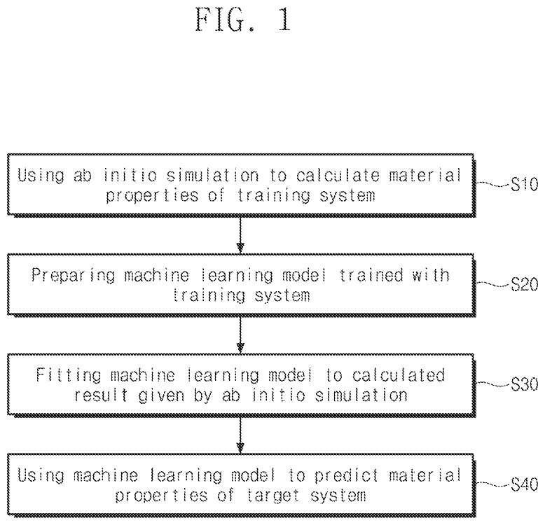

FIG. 1 illustrates a flow chart showing a method of predicting semiconductor material properties according to some embodiments of the present inventive concepts.

Referring to FIG. 1, a method of predicting semiconductor material properties according to the present inventive concepts may include using an ab initio simulation to calculate material properties of a training system (S10), preparing a machine learning model that is trained with the training system (S20), fitting the machine learning model to calculated results given by or outputs of the ab initio simulation (S30), and using the machine learning model to predict material properties of the target system (S40). The number (i.e., the quantity) of atoms included in the target system may be greater than or equal to the number of atoms included in the training system. For example, the target system may include about 100 or more single atoms, in some embodiments about 1,000 or more single atoms.

A descriptor, as discussed below, may be calculated from unrelaxed charge density (UCD) given by summation of atomic charge density (ACD) of single atoms.

For example, density functional theory (DFT) may be employed as the ab initio simulation. The operation S20 of preparing a machine learning model that is trained with the training system may include, for example, preparing a plurality of training data obtained from random displacement of the atoms included in the training system, extracting a descriptor for each of the plurality of training data, and training the machine learning model that is represented as or by a function of material properties with respect to the descriptor. The training data obtained from random displacement of the atoms included in the training system may be about 1,000 or more.

The extraction of the descriptor may include, for example, summing up the atomic charge density (ACD) to calculate the unrelaxed charge density (UCD), processing the unrelaxed charge density (UCD) to calculate an electron radial distribution function (ERDF), allowing grid lines to equally divide a horizontal axis of a graph that represents the electron radial distribution function (ERDF), and extracting a vector whose components are function values of grid points where the grid lines meet the electron radial distribution function (ERDF).

The step S30 of fitting the machine learning model to calculated results given by the ab initio simulation may include reducing or minimizing a difference between a calculated result obtained from the machine learning model and a calculated result obtained from the ab initio simulation.

For example, an algorithm of the machine learning model may be one or more of neural network (NN), convolutional neural network (CNN), graph neural network (GNN), and Gaussian process regression (GPR).

The method of predicting semiconductor material properties according to the present inventive concepts may predict material properties of large scale systems including semiconductor materials with increased speed and accuracy. In addition, the method of predicting semiconductor material properties may be employed to test semiconductor devices, such DRAM or NAND Flash or other memory devices.

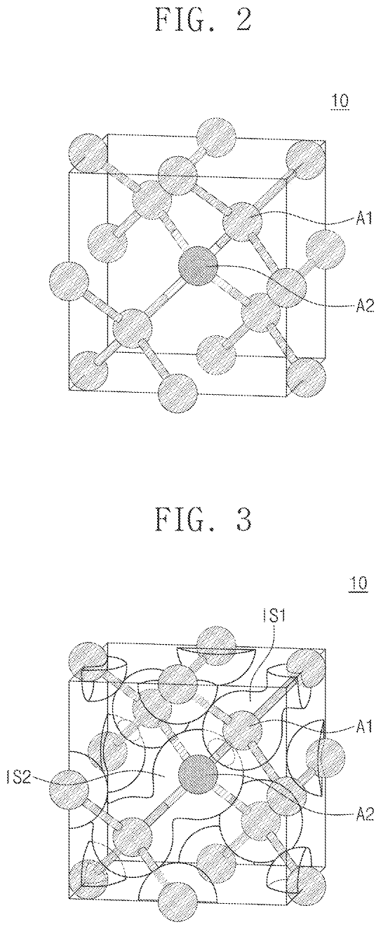

FIGS. 2 and 3 illustrate conceptual diagrams showing a training system for a method of predicting semiconductor material properties according to some embodiments of the present inventive concepts.

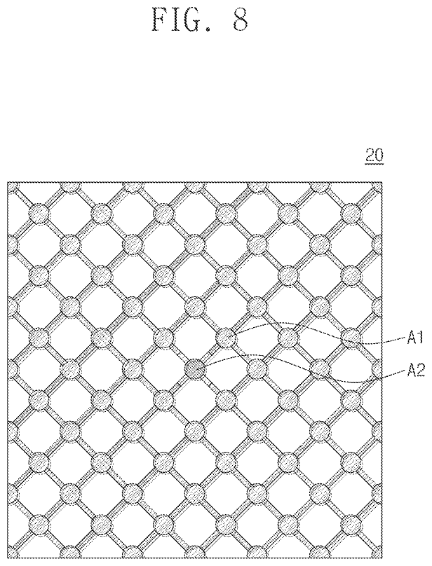

Referring to FIG. 2, a training system 10 may include, for example, about eight single atoms. The present inventive concepts, however, are not limited to this example embodiment, and the training system 10 may be a small scale system in which the ab initio simulation (e.g., density functional theory (DFT)) is used for calculation. For example, the training system 10 may include a number or quantity of single atoms less than or equal to that of single atoms included in a target system which will be discussed below (see 20 of FIG. 8).

For example as shown in FIG. 2, the training system 10 may include about seven first atoms A1 and about one second atom A2. For example, each of the first atoms A1 may be silicon (Si), and the second atom A2 may be one of germanium (Ge), phosphorus (P), boron (B), phosphorous cation (P⁺), or boron anion (B⁻). The second atom A2 may indicate an impurity or defect in a semiconductor material.

Referring to FIG. 3, first and second iso-surfaces IS1 and IS2 may be illustrated to represent fictitious or simulated electron distributions around the first and second atoms A1 and A2. The first and second iso-surfaces IS1 and IS2 may be, for example, iso-surfaces of the unrelaxed charge density (UCD). The methods of predicting semiconductor material properties according to the present inventive concepts may use a descriptor calculated from the unrelaxed charge density (UCD) that is represented as summation of the atomic charge density (ACD) of single atoms.

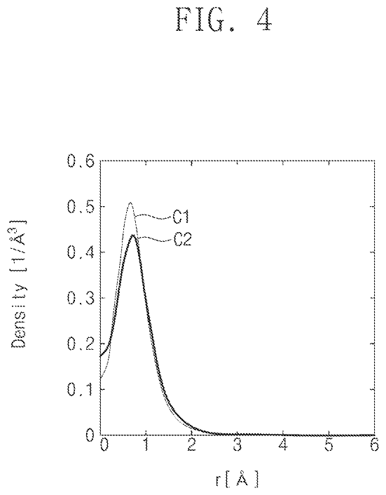

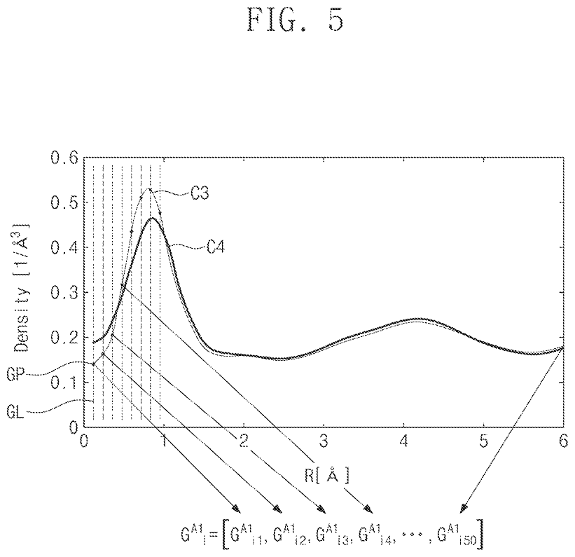

FIGS. 4 and 5 illustrate graphs showing a calculation procedure in a method of predicting semiconductor material properties according to some embodiments of the present inventive concepts. For example, FIG. 4 depicts a graph that represents the atomic charge density (ACD) of single atom, and FIG. 5 depicts a graph that represents the electron radial distribution function (ERDF) calculated from the unrelaxed charge density (UCD) that is represented as summation of the atomic charge density (ACD) of single atoms. In FIGS. 4 and 5, a horizontal axis may have a unit of Å, and a vertical axis may have a unit of 1/Å³.

Atomic charge density, nₐ(r), of single atom may be a solution of one-dimensional Kohn-Sham equation given by Mathematical Equation 1 below, and may be represented by Mathematical Equation 2 below.

$$\begin{matrix}[ \frac{- 1}{2} \frac{{d}^{2}}{d {r}^{2}} + \frac{l ( l + 1 )}{2 {r}^{2}} + V [ {n}_{a} ; r ] ] r {R}_{nl} ( r ) = {ɛ}_{n l} r {R}_{nl} ( r ) & [ Mathematical Equation 1 ] \\ {n}_{a} ( r ) = {\sum }_{nl}^{occ} {\vert {R}_{nl} ( r ) \vert}^{2} & [ Mathematical Equation 2 ]\end{matrix}$$

wherein, r is a radial distance from the center of a reference atom, n is a principal quantum number, 1 is an orbital quantum number (or angular quantum number), V[nₐ;r] is an exchange-correlation potential, and Rₙₗ(r) is a radial part of the wave function.

The atomic charge density nₐ(r) of single atom may be defined as summation of the absolute value of Rₙₗ(r) until an occupied state occ. The exchange-correlation potential V[nₐ;r] may be a functional of the atomic charge density nₐ(r) of single atom, and may be approximately calculated by local density approximation (LDA) or generalized gradient approximation (GGA).

Additionally, in order to reduce computational cost, pseudo charge density nₐ^{ps}(r) may be calculated by using pseudo wave function R^{ps}ₙₗ(r) and cut-off radius that is determined by a kind of atom or element. For example, the cut-off radii of silicon (Si), germanium (Ge), and phosphorus (P) may be about 1.00 Å, 1.27 Å, and 1.00 Å, respectively, and 2s and 2p of boron (B) may be about 0.79 Å and 0.89 Å, respectively.

The pseudo charge density nₐ^{ps}(r) may be expressed by Mathematical Equation 3 below, and may be defined as summation of the square of the absolute value of R^{ps}ₙₗ(R) for valence electrons.

$${{n}_{a}}^{ps} ( r ) = {\sum }_{nl}^{valence} {\vert {{R}^{ps}}_{nl} ( r ) \vert}^{2} \qquad [ Mathematical Equation 3 ]$$

Referring to FIGS. 3 and 4, a first curve C1 and a second curve C2 may indicate pseudo charge densities, calculated by the ways discussed above, of the first atom A1 and the second atom A2, respectively. The graph of FIG. 4 may show pseudo charge density nₐ^{ps}(r) of a Si₇Se system in which the first atom A1 is silicon (Si) and the second atom A2 is germanium (Ge). The first curve C1 and the second curve C2 may exhibit a certain difference when the radial distance r is less than about 3 Å, but may all converge to zero when the radial distance r is greater than about 3 Å. The difference between the first curve C1 and the second curve C2 may reflect a difference in distribution of four valence electrons in the silicon (Si) and germanium (Ge).

The unrelaxed charge density (UCD), n(r), may be expressed by Mathematical Equation 4 below that is summation of the pseudo charge density nₐ^{ps}(r) discussed with reference to Mathematical Equation 3 and FIG. 4.

$$n ( r ) = {\sum }_{j = 1}^{N} {{n}_{a}}^{ps} ( \vert r - {r}_{j} \vert ) \qquad [ Mathematical Equation 4 ]$$

wherein, N is the number of atoms in a sphere having a cut-off radius.

The unrelaxed charge density (UCD), n(r), may be considered as an unrelaxed charge density because of ignorance of relaxation of electron distribution due to the presence of other atoms. That is, the unrelaxed charge density (UCD) may not account for effects of other atoms on electron distribution.

The unrelaxed charge density (UCD), n(r), may satisfy neither rotational symmetry nor translational symmetry, and thus may not be directly used as a descriptor for machine learning model. Therefore, a machine learning model for the method of predicting semiconductor material properties according to the present inventive concepts may use a descriptor calculated from the unrelaxed charge density (UCD), n(r).

The descriptor may be, for example, an electron radial distribution function (ERDF), Gᵢ(r), given by Mathematic Equation 5 below. The present inventive concepts, however, are not limited thereto, and the descriptor may be other functions that satisfy the rotational symmetry and the translational symmetry.

$${G}_{i} ( R ) = \frac{\begin{matrix}{\int }_{R - Δ \frac{r}{2}}^{R + Δ \frac{r}{2}} {\int }_{0}^{} {\int }_{0}^{2 } \\ n ( r - {r}_{i} ) d r rd θ r sin θ d φ\end{matrix}}{\begin{matrix}{\int }_{R - Δ \frac{r}{2}}^{R + Δ \frac{r}{2}} {\int }_{0}^{} {\int }_{0}^{2 } \\ d r rd θ r sin θ d φ\end{matrix}} ∼ \frac{1}{4 } {\int }_{0}^{} {\int }_{0}^{2 } n ( R - {r}_{i} ) d θ sin θ d φ \qquad [ Mathematical Equation 5 ]$$

wherein, R is a vector expressed by (R sin θ cos φ, R sin θ sin φ, R cos θ).

The electron radial distribution function (ERDF), Gᵢ(r), may be a result of integrating the unrelaxed charge density (UCD), n(r), with respect to a spherical coordinate system in which an i^{th} atom is set to be an origin. In this sense, the electron radial distribution function (ERDF), Gᵢ(r), may be a function calculated from the unrelaxed charge density (UCD), n(r). The electron radial distribution function (ERDF), Gᵢ(r), may be a symmetric function and may be used as the descriptor for machine learning model.

Referring to FIGS. 3 and 5, a third curve C3 may indicate an electron radial distribution function (ERDF), Gᵢ(r), in which the first atom A1 is set to be an origin, and a fourth curve C4 may indicate an electron radial distribution function (ERDF), Gᵢ(r), in which the second atom A2 is set to be an origin. For example, the graph of FIG. 5 may show an electron radial distribution function (ERDF), Gᵢ(r), of a Si₇Se system in which the first atom A1 is silicon (Si) and the second atom A2 is germanium (Ge). The third curve C3 and the fourth curve C4 may exhibit a certain difference when R is less than about 2 Å, but may have substantially the same tendency when R is greater than about 2 Å. The difference between the third curve C3 and the fourth curve C4 when R is less than about 2 Å may reflect a difference in local charge density.

The descriptor of machine learning model may be expressed by first and second vectors G^{A1}ᵢ and G^{A2}ᵢ whose components are function values of 50 grid points GP where 50 grid lines GL meet one electron radial distribution function (ERDF), Gᵢ(r). In this case, the 50 grid lines GL may equally divide a horizontal axis of the graph shown in FIG. 5. The number of the grid lines GL and the number of the grid points GP are only provided by way of example, and the present inventive concepts are not limited thereto. In addition, the number of the grid lines GL and the number of the grid points GP may not depend on any properties of constituent elements.

The grid points GP of FIG. 5 may denote, for example, interconnections between the third curve C3 and the grid lines GL. The first vector G^{A1}ᵢ may represent a vector whose components are function values G^{A1}ᵢ₁, G^{A1}ᵢ₂, . . . , and G^{A1}ᵢ₅₀ of the grid points GP where the grid lines GL meet the third curve C3, and the second vector G^{A2}ᵢ may represent a vector whose components are function values of the grid points GP where the grid lines GL meet the fourth curve C4.

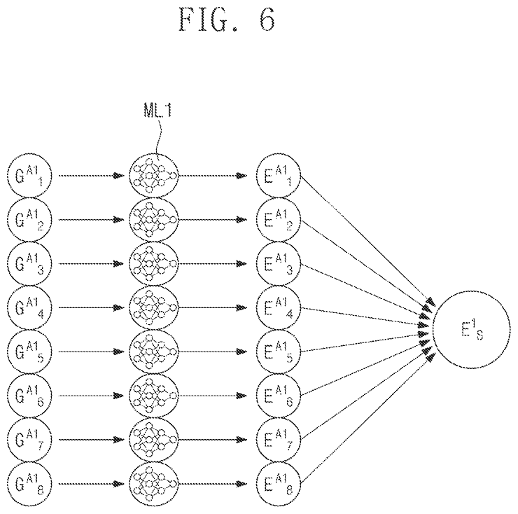

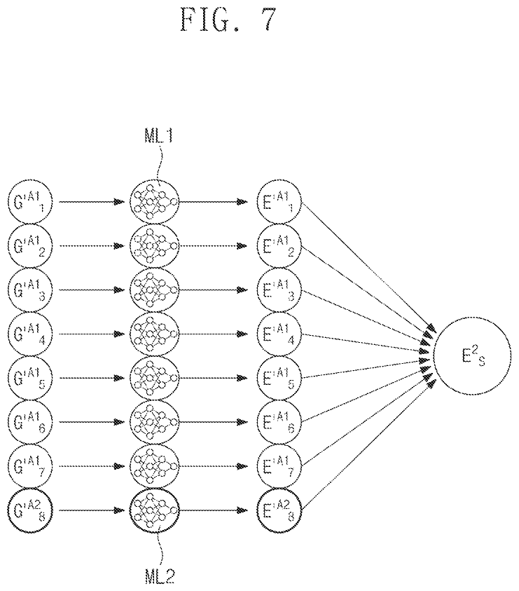

FIGS. 6 and 7 illustrate conceptual diagrams showing operations for preparing a machine learning model for a method of predicting semiconductor material properties according to some embodiments of the present inventive concepts. For example, FIG. 6 depicts a conceptual diagram showing a machine learning model for first training data including eight identical atoms (e.g., silicon (Si) atoms), and FIG. 7 depicts a conceptual diagram showing a machine learning model for second training data including seven identical atoms (e.g., silicon (Si) atoms) and one different atom (e.g., germanium (Ge) atom).

Referring to FIG. 6, a first machine learning model ML1 may learn a correlation between material properties E^{A1}₁, E^{A1}₂, . . . , and E^{A1}₈ of the first training data and descriptor vectors G^{A1}₁, G^{A1}₂, . . . and G^{A1}₈ expressed by the method discussed with reference to FIG. 5. Average material properties E¹s of the first training data may be defined as an average of the material properties E^{A1}₁, E^{A1}₂, . . . , and E^{A1}₈.

In this description, “material properties” may include total energy, binding energy, elastic constant, and dielectric constant of a system, and may also include atomic forces in the system.

In FIG. 6, the descriptor vectors G^{A1}₁, G^{A1}₂, . . . , and G^{A1}₈ may be calculated for the same atom (e.g., silicon (Si) atom), and may each be input to the first machine learning model ML1.

Referring to FIG. 7, a first machine learning model ML1 or a second machine learning model ML2 may learn a correlation between material properties E′^{A1}₁, E′^{A1}₂, . . . , E′^{A1}₇, and E′^{A2}₈ of the second training data and descriptor vectors G′^{A1}₁, G′^{A1}₂, . . . , G′^{A1}₇ and G′^{A2}₈ expressed by the method discussed with reference to FIG. 5. The second machine learning model ML2 may be different from the first machine learning model ML1. Average material properties E²s of the second training data may be defined as an average of the material properties E′^{A1}₁, E′^{A1}₂, . . . E′^{A1}₇, and E′^{A2}₈.

In FIG. 7, among the descriptor vectors G′^{A1}₁, G′^{A1}₂, . . . , G′^{A1}₇, and G′^{A2}₈, seven descriptor vectors G′^{A1}₁, G′^{A1}₂, . . . , and G′^{A1}₇ may be calculated for the same atom (e.g., silicon (Si) atom), and may each be input to the first machine learning model ML1. Among the descriptor vectors G′^{A1}₁, G′^{A1}₂, . . . , G′^{A1}₇, and G′^{A2}₈, one descriptor vector G′^{A2}₈ may be calculated for a different atom (e.g., germanium (Ge) atom), and may be input to the second machine learning model ML2.

The first machine learning model ML1 and the second machine learning model ML2 may include layers (e.g., input layers, hidden layer, and output layers) each including a plurality of nodes. The nodes included in each of the layers may be connected to each other through a weight. The weight may be set based on the preparation of machine learning model. The weight that connects the layers of the first machine learning model ML1 may be different from the weight that connects the layers of the second machine learning model ML2.

FIG. 8 illustrates a conceptual diagram showing a target system for a method of predicting semiconductor material properties according to some embodiments of the present inventive concepts.

Referring to FIG. 8, a target system 20 may include, for example, about 64 single atoms. The present inventive concepts, however, are not limited to this example embodiment, and the target system 20 may be a large scale system in which the ab initio simulation accelerated by machine learning according to the present inventive concepts is used for prompt and accurate calculation. In this sense, the target system 20 may include single atoms the number of which is the same as or greater than that of the single atoms included in the aforementioned training system (see 10 of FIGS. 2 and 3). For example, the target system 20 may include about 100 or more single atoms, preferably about 1,000 or more single atoms.

The target system 20 of FIG. 8 may include, for example, about 63 first atoms A1 and about one second atom A2. For example, the first atoms A1 may each be silicon (Si), and the second atom A2 may be one of germanium (Ge), phosphorus (P), boron (B), phosphorous cation (P⁺), and boron anion (B⁻). The second atom A2 may indicate an impurity or defect in a semiconductor material.

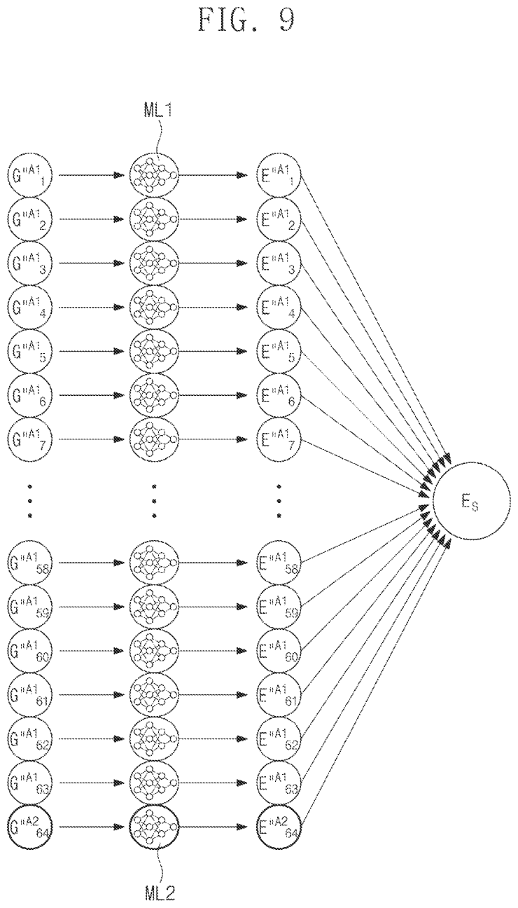

FIG. 9 illustrates a conceptual diagram showing prediction of material properties of a target system with the help of machine learning model for a method of predicting semiconductor material properties according to some embodiments of the present inventive concepts.

Referring to FIG. 9, descriptor vectors G″^{A1}₁, G″^{A1}₂, . . . , G″^{A1}₆₃, and G″^{A2}₆₄ expressed by the method discussed with reference to FIG. 5 may each be input to the first machine learning model ML1 or the second machine learning model ML2, and may be output as predictions E″^{A1}₁, E″^{A1}₂, . . . , E″^{A1}₆₃, and E″^{A2}₆₄, which are associated with material properties included in a target system, from the first machine learning model ML1 and/or the second machine learning model ML2. An average prediction Eₛ of material properties in a target system may be calculated from an average of the material property predictions E″^{A1}₁, E″^{A1}₂, . . . , E″^{A1}₆₃, and E″^{A2}₆₄.

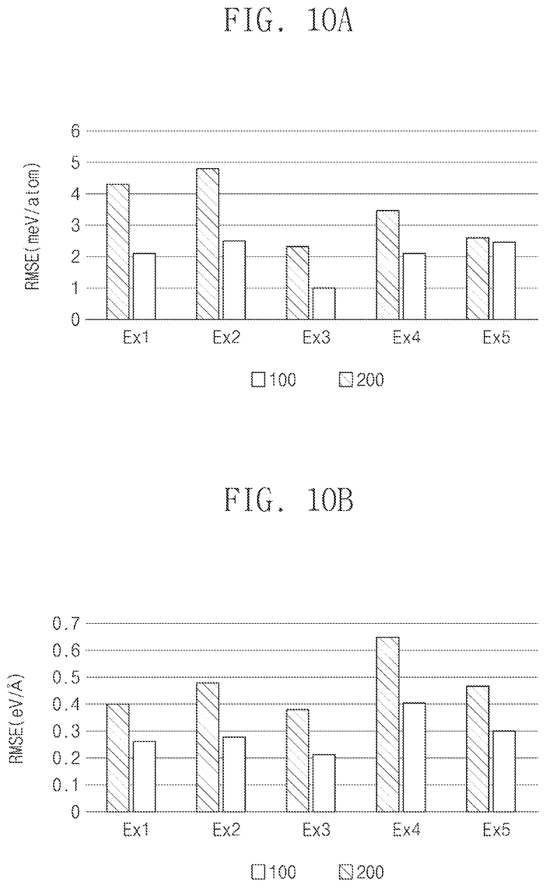

FIGS. 10A, 10B, 11A, 11B, 12A, and 12B illustrate graphs showing an error in case of using a descriptor for a method of predicting semiconductor material properties according to some embodiments of the present inventive concepts. In description below, the term “error” may refer to a root mean square RMSE. The expression “meV/atom” may be used as a unit of the error in FIGS. 10A, 11A, and 12A, and the expression “eV/Å” may be used as a unit of the error in FIGS. 10B, 11B, and 12B.

For example, the graphs of FIGS. 10A, 10B, 11A, 11B, 12A, and 12B may show the comparison between a case 100 where the electron radial distribution function (ERDF) is used as a descriptor according to some embodiments of the present inventive concepts and a case 200 where a descriptor that is calculated by an atomic number, an inter-atomic distance, and an angular term defined between three atoms is used according to a comparative example.

Separately from the following experimental examples, about 40 hours may be consumed in a case that the ab initio simulation (e.g., density functional theory (DFT)) is used to calculate material properties of a target system including about 216 atoms, but about 12 hours may be consumed in a case that a machine learning model is trained with a training system including about eight atoms, and then the trained machine learning model is applied to predict material properties of a target system including about 216 atoms. In this sense, when using the method of predicting semiconductor material properties according to some embodiments of the present inventive concepts, it may be possible to predict material properties promptly or more quickly, compared to a case in which the ab initio simulation is used.

Moreover, because a time required for the ab initio simulation increases in proportion to the cube of the number of atoms, and because a time required for the method of predicting semiconductor material properties according to some embodiments of the present inventive concepts increases in proportion to the number of atoms, the method of predicting semiconductor material properties according to some embodiments of the present inventive concepts may be more appropriate to and more efficient for material property predictions for large scale systems.

FIGS. 10A and 10B depict graphs showing an error of material property prediction for a system including germanium-doped silicon (Si) atoms. For example, FIG. 10A shows a case in which total energy is predicted, and FIG. 10B shows a case in which atomic forces are predicted.

A first experimental example Ex1 may be a case in which a machine learning model is trained with a training system including eight silicon (Si) atoms, and then the trained machine learning model is applied to predict material properties of a target system including eight silicon (Si) atoms.

A second experimental example Ex2 may be a case in which a machine learning model is trained with a training system including seven silicon (Si) atoms and one germanium (Ge) atom, and then the trained machine learning model is applied to predict material properties of a target system including seven silicon (Si) atoms and one germanium (Ge) atom.

A third experimental example Ex3 may be a case in which a machine learning model is trained with a training system including 63 silicon (Si) atoms and one germanium (Ge) atom, and then the trained machine learning model is applied to predict material properties of a target system including 63 silicon (Si) atoms and one germanium (Ge) atom.

A fourth experimental example Ex4 may be a case in which a machine learning model is trained with a training system including eight silicon (Si) atoms and a training system including seven silicon (Si) atoms and one germanium (Ge) atom, and then the trained machine learning model is applied to predict material properties of a target system including 63 silicon (Si) atoms and one germanium (Ge) atom.

A fifth experimental example Ex5 may be a case in which a machine learning model is trained with a training system including eight silicon (Si) atoms, a training system including seven silicon (Si) atoms and one germanium (Ge) atom, and a training system including 15 silicon (Si) atoms and one germanium (Ge) atom, and then the trained machine learning model is applied to predict material properties of a target system including 63 silicon (Si) atoms and one germanium (Ge) atom.

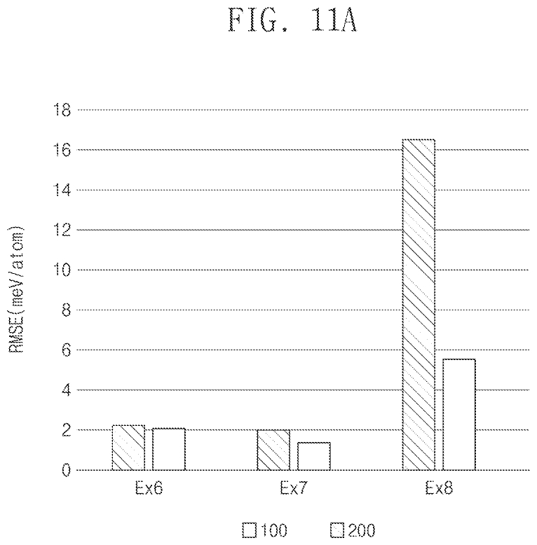

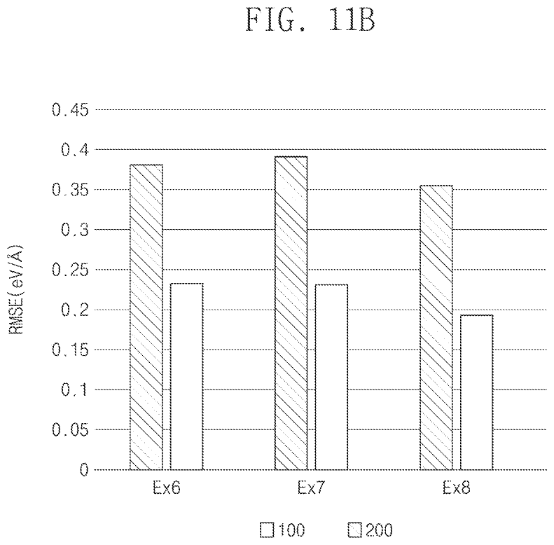

FIGS. 11A and 11B depict graphs showing an error of material property prediction for a system including phosphorus-doped or phosphorous cation-doped silicon (Si) atoms. For example, FIG. 11A shows a case in which total energy is predicted, and FIG. 11B shows a case in which atomic forces are predicted.

A sixth experimental example Ex6 may be a case in which a machine learning model is trained with a training system including 63 silicon (Si) atoms and one phosphorus (P) atom, and then the trained machine learning model is applied to predict material properties of a target system including 63 silicon (Si) atoms and one phosphorus (P) atom.

A seventh experimental example Ex7 may be a case in which a machine learning model is trained with a training system including 63 silicon (Si) atoms and one phosphorous cation (P⁺), and then the trained machine learning model is applied to predict material properties of a target system including 63 silicon (Si) atoms and one phosphorous cation (P⁺).

An eighth experimental example Ex8 may be a case in which a machine learning model is trained with a training system including 64 silicon (Si) atoms, a training system including 63 silicon (Si) atoms and one phosphorus (P) atom, and a training system including 63 silicon (Si) atoms and one phosphorous cation (P⁺), and then the trained machine learning model is applied to predict material properties of a target system including 215 silicon (Si) atoms and one phosphorus (P) atom (or one phosphorous cation (P⁺)).

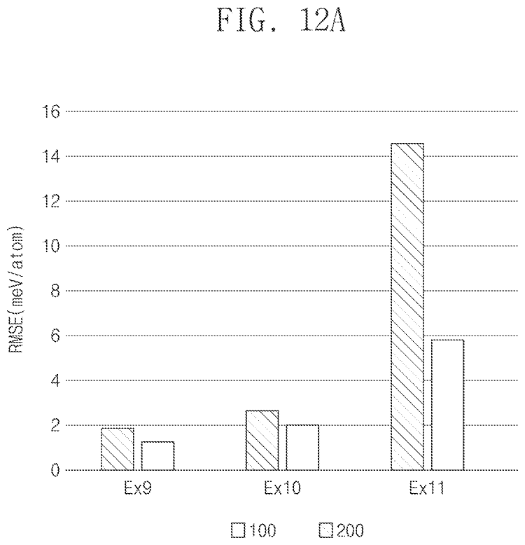

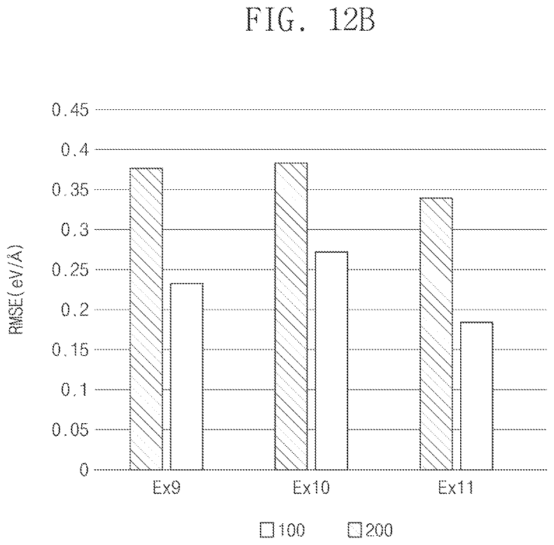

FIGS. 12A and 12B depict graphs showing an error of material property prediction for a system including boron-doped or boron anion-doped silicon (Si) atoms. For example, FIG. 12A shows a case in which total energy is predicted, and FIG. 12B shows a case in which atomic forces are predicted.

A ninth experimental example Ex9 may be a case in which a machine learning model is trained with a training system including 63 silicon (Si) atoms and one boron (B) atom, and then the trained machine learning model is applied to predict material properties of a target system including 63 silicon (Si) atoms and one boron (B) atom.

A tenth experimental example Ex10 may be a case in which a machine learning model is trained with a training system including 63 silicon (Si) atoms and one boron anion (B⁻), and then the trained machine learning model is applied to predict material properties of a target system including 63 silicon (Si) atoms and one boron anion (B⁻).

An eleventh experimental example Ex11 may be a case in which a machine learning model is trained with a training system including 64 silicon (Si) atoms, a training system including 63 silicon (Si) atoms and one boron (B) atom, and a training system including 63 silicon (Si) atoms and one boron anion (B⁻) atom, and then the trained machine learning model is applied to predict material properties of a target system including 215 silicon (Si) atoms and one boron (B) atom (or one boron anion (B⁻)).

In all of the first to eleventh experimental examples Ex1 to Ex11, it may be ascertained that an error in the case 100 where the electron radial distribution function (ERDF) is used as a descriptor according to some embodiments of the present inventive concepts is less than an error in the case 200 according to a comparative example. In brief, the case 100 in which the electron radial distribution function (ERDF) is used as a descriptor according to some embodiments of the present inventive concepts may predict material properties more accurately than the comparative case 200.

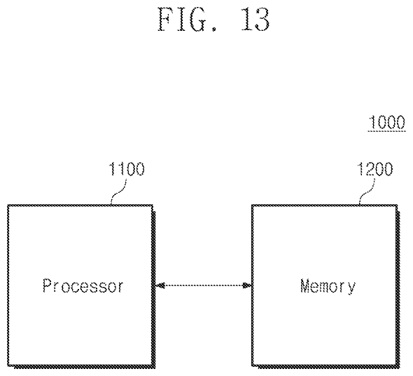

FIG. 13 illustrates a block diagram showing a computing system related to a method of predicting semiconductor material properties according to some embodiments of the present inventive concepts.

Referring to FIG. 13, a computing system 1000 may include a processor 1100 and a memory 1200. The computing system 1000 may be configured to drive or otherwise perform a method of predicting semiconductor material properties according to some embodiments of the present inventive concepts.

The memory 1200 may include commands that can be executed by the processor 1100 to perform one or more operations as described herein. For example, the memory 1200 may include a machine learning model that is prepared as discussed with reference to FIGS. 6 and 7. The processor 1100 may execute the commands stored in the memory 1200 and thus may perform the aforementioned method of predicting semiconductor material properties. The memory 1200 may store results obtained from operations of the processor 1100, and the results stored in the memory 1200 may be used to predict properties of semiconductor materials that correspond to a target system, with the result that a semiconductor device may be tested.

A method of predicting semiconductor material properties according to some embodiments of the present inventive concepts may use an ab initio simulation that is accelerated by machine learning to thereby improve speed and accuracy in predicting material properties for large scale system.

Although the present invention has been described in connection with the some example embodiments of the present inventive concepts illustrated in the accompanying drawings, it will be understood by one of ordinary skill in the art that variations in form and detail may be made therein without departing from the spirit and essential feature of the present inventive concepts. The above disclosed embodiments should thus be considered illustrative and not restrictive.

What is claimed is:

1. A method of predicting semiconductor material properties, the method comprising:
executing, by at least one processor, commands stored in a non-transitory memory to perform operations comprising:
accessing a machine learning model that is trained with a training system, wherein the training system comprises calculated semiconductor material properties; and
using the machine learning model to predict material properties of a target system comprising one or more semiconductor materials,
wherein the machine learning model is represented by a function of material properties with respect to a descriptor, and
wherein the descriptor is calculated from unrelaxed charge density (UCD) that is represented by summation of atomic charge density (ACD) of single atoms.

2. The method of claim 1, wherein a number of atoms in the target system is greater than or equal to a number of atoms in the training system.

3. The method of claim 1, wherein the descriptor comprises a function that satisfies rotational symmetry and translational symmetry.

4. The method of claim 1, wherein the atomic charge density (ACD) is a solution of one-dimensional Kohn-Sham equation.

5. The method of claim 1, wherein the descriptor is an electron radial distribution function (ERDF).

6. The method of claim 5, wherein the electron radial distribution function (ERDF) is calculated by integrating the unrelaxed charge density (UCD) with respect to a spherical coordinate system.

7. The method of claim 1, wherein an algorithm of the machine learning model comprises neural network (NN), convolutional neural network (CNN), graph neural network (GNN), and/or Gaussian process regression (GPR).

8. The method of claim 1, wherein the material properties of the target system comprise total energy of the target system and atomic forces in the target system.

9. The method of claim 1, wherein the target system comprises a portion of a semiconductor device, and wherein the operations further comprise testing the semiconductor device based on the material properties that were predicted for the target system by the machine learning model.

10. A method of predicting semiconductor material properties, the method comprising:
executing, by at least one processor, commands stored in a non-transitory memory to perform operations comprising:
accessing a machine learning model that is trained with a training system, wherein the training system comprises calculated semiconductor material properties generated by an ab initio simulation;
fitting the machine learning model to an output of the ab initio simulation; and
using the machine learning model to predict material properties of a target system comprising one or more semiconductor materials,
wherein the machine learning model is represented by a function of material properties with respect to a descriptor,
wherein the descriptor is calculated from unrelaxed charge density (UCD).

11. The method of claim 10, wherein a number of atoms in the target system is greater than or equal to a number of atoms in the training system.

12. The method of claim 10, further comprising:
preparing a plurality of training data obtained by random displacement of atoms included in the training system;
extracting the descriptor for each of the training data; and
training the machine learning model.

13. The method of claim 12, wherein extracting the descriptor comprises:
calculating the unrelaxed charge density (UCD) by summation of atomic charge density (ACD) of single atoms;
processing the unrelaxed charge density (UCD) to calculate an electron radial distribution function (ERDF); and
extracting a vector whose components are function values of grid points where grid lines meet the electron radial distribution function (ERDF), wherein the grid lines equally divide a horizontal axis of a graph that represents the electron radial distribution function (ERDF).

14. The method of claim 13, wherein the electron radial distribution function (ERDF) is calculated by integrating the unrelaxed charge density (UCD) with respect to a spherical coordinate system.

15. The method of claim 10, wherein fitting the machine learning model to the output of the ab initio simulation comprises reducing a difference between a calculated result obtained from the machine learning model and a calculated result obtained from the ab initio simulation.

16. The method of claim 10, wherein an algorithm of the machine learning model comprises neural network (NN), convolutional neural network (CNN), graph neural network (GNN), and/or Gaussian process regression (GPR), and wherein the material properties of the target system comprise total energy, binding energy, elastic constant, and dielectric constant of the target system, and atomic forces in the target system.

17. The method of claim 10, wherein the target system comprises a portion of a semiconductor device, and wherein the operations further comprise testing the semiconductor device based on the material properties that were predicted for the target system by the machine learning model.

18. A method of testing a semiconductor device, the method comprising:
executing, by at least one processor, commands stored in a non-transitory memory to perform operations comprising:
accessing a machine learning model that is trained with a training system, wherein the training system comprises calculated semiconductor material properties generated by density functional theory (DFT);
fitting the machine learning model to a result calculated from the density functional theory (DFT);
using the machine learning model to predict material properties of a target system that is a portion of the semiconductor device; and
testing the semiconductor device based on the material properties that were predicted for the target system by the machine learning model,
wherein the machine learning model is represented by a function of material properties with respect to a descriptor,
wherein the descriptor is calculated from unrelaxed charge density (UCD) that is represented by summation of atomic charge density (ACD) of single atoms.

19. The method of claim 18, wherein a number of atoms in the target system is greater than or equal to a number of atoms in the training system.

20. The method of claim 18, wherein the descriptor is an electron radial distribution function (ERDF).
